(19) United States
(12) Patent Application Publication
(10) Pub. No.: US 2012/0181201 A1
(43) Pub. Date: Jul. 19, 2012

(54) Topical Formulation Containing a Tetracycline and a Method of Treating Skin Infections Using the Same

(75) Inventor: William Heggie, Cabanas (PT)

(73) Assignee: HOVIONE INTER LIMITED, Lucerne 7 (CH)

(21) Appl. No.: 13/380,283

(22) PCT Filed: Jun. 25, 2010
(86) PCT No.: PCT/GB10/01251

§ 371 Date: Jan. 26, 2012

(30) Foreign Application Priority Data
Jun. 26, 2009 (PT) PT104644

(51) Int. Cl. A61K 31/65; A61P 17/10; A61Q 17/04; B65D 85/00; A61Q 19/00; A61P 31/04; A61K 8/67; A61P 29/00; A61K 8/42; A61Q 19/08

(52) U.S. Cl. 206/438; 514/152; 514/153; 514/154; 424/59

(57) ABSTRACT

A topical formulation comprising a tetracycline comprises two separate parts: (i) a first part comprising a tetracyline in solid form suspended in a first vehicle; and (ii) a second part comprising a second vehicle in which the tetracycline is soluble. Preferably the tetracycline is crystalline minocycline base. Suitably, a neutral vehicle is used for the first part of the formulation. The two parts of the formulation may be packaged in separate containers and are preferably topically applied therefrom simultaneously.

This invention relates to a topical formulation comprising a tetracycline, particularly minocycline base, and to a method of treating skin infections using the same. Until now, minocycline base, 7-dimethylamino-6-deoxy-6-dimethyltetracycline, was known in the form of an amorphous solid, which is inherently unstable, not only in solution but even when solid.

Crystalline forms of minocycline base were first described in the Portuguese patent application PT103661, corresponding to international patent application WO2008/102161. The amorphous form of minocycline, known until then was isolated by evaporating from a solution, an extensive degradation occurring during this process giving rise to 4-epi-minocycline, which is a compound without antibacterial activity.

The invention of the Portuguese patent application PT103661 allows the preparation of minocycline in a highly purified form, which can then be crystallized giving rise to different polymorphs.

Minocycline is used to treat skin infections, usually acne, in the form of the hydrochloride, which is administered orally and acts in a systemic fashion. Topical administration on the infected area would be highly advantageous as the amount of active ingredient used in the treatment can be reduced, since it is administered directly to the site of infection.

Minocycline hydrochloride has been subject to several topical formulations (e.g. one formulation is described in European patent EP0410099 A1). Although this salt is more stable in solution than the corresponding base it presents the disadvantage, as it is an acid addition salt, of having an acid pH causing increased skin irritation, especially if the application is made on an infected area.

Formulations containing minocycline base, where the base is in solution, are inherently unstable. This happens not only in the case of aqueous solvents but also when the solvent is organic.

However, minocycline base is easily transportable through the epidermis, due to its high solubility in lipids, whereas minocycline in the form of its acid addition salts (e.g. hydrochloride), is more difficult to transport to the place of action, is acidic and is more aggressive for the skin.

According to one aspect of the present invention, there is provided a topical formulation comprising a tetracycline characterized in that the formulation comprises two separate parts:
(i) a first part comprising a tetracycline in solid form suspended in a first vehicle; and
(ii) a second part comprising a second vehicle in which the tetracycline is soluble.

In another aspect, there is provided a formulation comprising crystalline minocycline base in solid form and a pharmaceutically acceptable vehicle in which said minocycline is insoluble.

In another aspect, there is provided a kit of parts comprising;
(i) a first part comprising a tetracycline in solid form suspended in a first vehicle; and
(ii) a second part comprising a second vehicle in which the tetracycline is soluble.

In a highly preferred aspect, the tetracycline is insoluble or substantially insoluble in the first vehicle.

By insoluble or substantially insoluble, we mean that essentially no material (or only trace amounts such as less than 1% of the active by weight) can be detected in solution by conventional analytical methods.

By soluble we mean that essentially no solid material (or only trace amounts, such as less than 1% of the active by weight) is detected using conventional analytical methods, after mixing of the active with the second vehicle.

Preferably, a crystalline form of the active material is used. One preferred active is crystalline minocycline base, which can be present if desired in one of the crystalline polymorphic forms (I, II or III) described in WO2008/102161, or as a mixture thereof.

The present invention benefits from the availability of, for example, minocycline base in an appropriately pure and stable form in the solid state. The instability of minocycline in solution is resolved by suspending minocycline base in a vehicle before being applied to the skin over the area of infection. Preferably, the vehicle is a neutral vehicle. Suitably, a neutral vehicle is one that does not substantially change the pH of the tetracycline (preferably within a tolerance of ±1 pH unit). Preferably, there is no change in pH upon mixing of the active and vehicle. A neutral vehicle is also one which does not react with the tetracycline. As the absorption of solid material through the skin's natural defensive barrier is extremely difficult, a second vehicle, which has the ability of dissolving or substantially dissolving minocycline and transporting the active ingredient to the site of infection is applied, preferably simultaneously, to the same area.

Suitably, the two parts of the formulation are prepared separately and are packed in separate containers. By way of example, but not by way of limitation, these can be bottles, tubes or roll-ons. The containers can be grouped in a package that integrates them in a way that both parts are applied simultaneously to the same area of skin being treated. Several examples of this type of package are known in the market.

This principle can be extended to cover salts of tetracyclines, such as minocycline hydrochloride. In this particular case of the invention, the vehicle in which the salt (e.g. hydrochloride) is insoluble may be the same as that used for minocycline base, but the second vehicle must be basic or buffered, in order to extemporaneously obtain minocycline base from minocycline hydrochloride.

The principle of the invention can also be extended to other antibiotics of the tetracycline family which have the same problems of stability in solution, either in their neutral base form or in the form of their acid addition salts.

Accordingly, where the salt of a tetracycline is used, particularly an acid addition salt, the second vehicle comprises a buffer or is of a basic pH. Suitable buffers will be known to those in the art.

Since it is known that tetracyclines may increase skin sensitivity to sunlight, either of the two vehicles may additionally contain a sunscreen, which offers the advantage of blocking sunlight to avoid this undesirable side effect.

Also, cosmetic and other additives may be used to help mitigate any localized skin reaction that may have resulted from the infection or treatment or improve the appearance of the skin in general.

If necessary, a second active substance can be added to any of the vehicles, provided it is compatible with the first active substance.

The invention preferably employs minocycline base in solid form (preferably crystalline form), which is suspended in a vehicle (first vehicle), in which minocycline base is insoluble or substantially insoluble, and therefore has the inherent stability of the solid crystalline form. Any liquid, gel or cream that is considered pharmaceutically acceptable for skin application and in which minocycline is insoluble may, for example, be used.

By way of example, preferred vehicles mainly used for the creation of the suspension are as follows: silicones, paraffin and mixtures thereof.

The concentration of minocycline in the suspension is not a particularly relevant factor since its absorption is limited by the permeability of the skin.

The second part of formulation (the second vehicle) is suitably chosen from pharmaceutically acceptable liquids, gels or creams in which minocycline base is soluble or substantially soluble. Among those that assist the transport of the active substance through the barriers of the skin are preferred.

By way of example, preferred vehicles used to solubilize the tetracycline such as minocycline are as follows: Transcutol (diethylene glycol ethyl ether), isopropyl myristate or mixtures thereof. However, other suitable solubilisers or permeation enhancers may be used.

Stabilizers and emulsifiers which are known and widely used in topical formulations may be added to each of the two parts of the formulation.

A generalised formulation is given in Table 1 below, together with examples of specific components which may be used. Note that the skin enhancer, emulsifying agent and stabilizing agent shown are optional features—one or more of these components may be present, or the formulation may just comprise active, first vehicle and second vehicle.

TABLE 1

| Example of specific | | Unit composition |
|---|---|---|
| components | General component | (g/200 g) |
| Minocycline base | Active substance | 2-4 |
| Paraffin, liquid | First vehicle | 60-90 |
| Paraffin | First vehicle | 38-8 |
| | SUB-TOTAL | 100 |
| Diethylene glycol | Second vehicle | 50-70 |
| monoethyl ether | | |
| Isopropyl myristate | Skin enhancer | 6-20 |
| Glyceryl monoestearate | Emulsifying agent | 16-20 |
| Linoleoyl | Stabilizing agent | 6-12 |
| macrogolglycerides | | |
| | SUB-TOTAL | 100 |
| | TOTAL | 200 |

In the particular case in which the tetracycline is minocycline hydrochloride, the first vehicle may be essentially the same as above but the second vehicle must contain a basic substance or buffer, in order to extemporaneously obtain minocycline base from minocycline hydrochloride upon mixing of the two parts on the skin.

It is known that when minocycline is used systemically, increased skin sensitivity to sunlight occurs. In the case of topical application this effect is limited to the area of application, and in that case a sunscreen can be simultaneously applied to protect the skin. The sunscreen may be added to any of the two vehicles. By way of example but not limited to, a preferred sunscreen is titanium dioxide, but any sunscreen suitable for use in pharmaceutical formulations can be used.

Additives with desirable cosmetic properties or others that help to mitigate any localized skin reaction that may result from infection or treatment or improve the appearance of the skin in general can be added to any of the vehicles. By way of example, but not by way of limitation, preferred cosmetic additives are moisturizers, antioxidants and or substances with soothing anti wrinkle and or anti spots effect.

One or more additional active ingredients may also be added to the formulation. By way of example, but not by way of limitation, other active ingredients may be substances with anti-inflammatory action, retinoids, vitamins (e.g. A, E) or other compounds which contribute to the improvement of the skin condition by helping the treatment of the infection.

Once prepared by mixing the various components of the formulation both parts of the formulation are packed in separate containers. By way of example, preferred containers are, but not limited to these, bottles, tubes or roll-ons. Containers can be of any material suitable for use in the pharmaceutical industry and compatible with the vehicles. Examples include but are not limited to, preferred materials such as glass, coated aluminum and plastics. The containers can be grouped in one applicator to allow a simultaneous application of both parts of the formulation. By way of example, the applicator may be an applicator of the type described in international application WO 2003/099295 or a compartmentalized tube. The two parts of the formulation are stable at room temperature for prolonged periods of time.

The application of the formulation is made by applying a drop of each of the formulation parts to the infected area, and gently massaging in order to mix the two parts. Consequently, the active (such as minocycline base) is dissolved and transported to the lower layers of the skin.

Other tetracyclines which have the same problems of stability in solution, either in their neutral base form or in the form of their acid addition salts may also benefit from the formulation of the present invention. By way of example, but not by way of limitation, we refer to tetracycline and doxycycline.

Besides the aforementioned advantages of a non-aggressive topical formulation for the treatment of acne, this invention provides a high permeability and a greater stability of the active agent as well as reducing the side effects caused by systemic administration.

### EXAMPLE

A formulation was made comprising the following components. Simple mixing of the ingredients was used for the suspension and the solubilizing vehicle.

Suspension
minocycline base 1%
Paraffin, liquid 40%
Paraffin 9%
   Partial total 50%

Solubilizing Vehicle
Diethylene glycol monoethyl ether 32%
Isopropyl myristate 5%
9% glyceryl monoestearate
linoleoyl macrogolglycerides of 4%
   Partial total 50%

Stability tests were carried out and after 30 days no significant epimerization was observed.

Percutaneous absorption studies done in vitro with human skin showed that after mixing the two parts of the formulation there was a permeation of minocycline base through the skin.

1. A topical formulation comprising a tetracycline characterized in that the formulation comprises two separate parts:
(i) a first part comprising a crystalline tetracycline base in solid form suspended in a first vehicle; and
(ii) a second part comprising a second vehicle in which the tetracycline is soluble;
characterized in that the second part is not basic or buffered.

2. A formulation according to claim 1 wherein the tetracycline is substantially insoluble or insoluble in the first vehicle.

3. A formulation according to claim 1 characterized in that the tetracycline is selected from tetracycline, doxycycline or minocycline.

4. A formulation according to claim 2 characterized in that the tetracycline is minocycline base.

5. A formulation according to claim 1 characterized in that the first vehicle is a pharmaceutical liquid, gel or cream acceptable for skin application, in which the tetracycline is substantially insoluble or insoluble.

6. A formulation according to claim 5 wherein the first vehicle comprises paraffin or silicone or a mixture thereof.

7. A formulation according to claim 1 wherein the second vehicle comprises a pharmaceutical liquid, gel or cream acceptable for skin application, in which the tetracycline is soluble.

8. A formulation according to claim 7 wherein the second vehicle comprises di(ethylene glycol) ethyl ether, isopropyl myristate or a mixture thereof.

9. A formulation according to claim 1 wherein the first part or the second part or both parts of the formulation comprises one or more stabilizers and/or one or more emulsifying substances.

10. A formulation according to claim 1 wherein the first or second vehicle or both vehicles comprises one or more additional active ingredients.

11. A formulation according to claim 10 wherein the additional active ingredient is selected from anti-inflammatory agents, retinoids, vitamins A, E, or mixtures thereof.

12. A formulation according to claim 1 wherein the first or second vehicle or both vehicles comprises one or more of any one or more of the following: a moisturizer; an antioxidant; a substance with soothing, anti wrinkle and/or anti-spots effect; or a sunscreen; or mixtures thereof.

13. A formulation according to claim 12 comprising titanium dioxide as sunscreen.

14. A formulation comprising crystalline minocycline base in solid form and a pharmaceutically acceptable vehicle in which said minocycline is substantially insoluble or insoluble.

15. A formulation according to claim 14 wherein the vehicle comprises a pharmaceutical liquid, gel, cream, paraffin or silicone or a mixture thereof, acceptable for skin application, in which the tetracycline is substantially insoluble or insoluble.

16. A formulation according to claim 14 further comprising one or more stabilizers, emulsifying substances; additional active ingredients, anti-inflammatory agents, retinoids, vitamins A, E, or mixtures thereof; moisturizers, antioxidants; substances with soothing, anti wrinkle and/or anti-spots effects; sunscreens; or mixtures thereof, and titanium dioxide.

17. A kit of parts which upon mixing of said parts provides a formulation suitable for topical delivery of a tetracycline, the kit comprising:
(i) a first part comprising a crystalline tetracycline base in solid form suspended in a first vehicle; and
(ii) a second part comprising a second vehicle in which the tetracycline is soluble;
characterized in that the second part is not basic or buffered.

18. A kit according to claim 17 wherein the first part is comprised of a crystalline tetracycline base substantially insoluble or insoluble in the first vehicle.

19. A container product comprising separate containers wherein a first container comprises the first part of the formulation as defined in claim 1, and a second container comprises the second part of the formulation as defined in claim 1.

20. A container product according to claim 19 wherein the first and second containers are integrated such that both parts of the formulation are delivered simultaneously from the said containers.

21. A container product according to claim 19 wherein the parts of the formulation can be delivered in a fixed amount and with a selected proportion of the first part to the second part.

22. A formulation according to claim 1 for use in the treatment of skin infections.

23. A formulation according to claim 22 characterized in that the skin infection is acne or rosacea.

24. The kit of claim 17, wherein the tetracycline substantially insoluble or insoluble in the first vehicle comprises one or more of: tetracycline, doxycycline or minocycline.

25. The kit of claim 17, wherein the tetracycline substantially insoluble or insoluble in the first vehicle comprises minocycline base.

26. The kit of claim 17, wherein the first vehicle comprises one or more of:
a pharmaceutical liquid, gel, cream, paraffin or silicone or a mixture thereof, acceptable for skin application, in which the tetracycline is substantially insoluble or insoluble;
and, optionally, one or more of: stabilizers, emulsifying substances; additional active ingredients, anti-inflammatory agents, retinoids, vitamins A, E, or mixtures thereof; moisturizers, antioxidants; substances with soothing, anti wrinkle and/or anti-spots effects; sunscreens; or mixtures thereof, and titanium dioxide.

27. The kit of claim 17, wherein the second vehicle comprises one or more of:
a pharmaceutical liquid, gel or cream or a mixture thereof, acceptable for skin application, in which the tetracycline is soluble;
and, optionally, one or more of: stabilizers, emulsifying substances; additional active ingredients, anti-inflammatory agents, retinoids, vitamins A, E, or mixtures thereof; moisturizers, antioxidants; substances with soothing, anti wrinkle and/or anti-spots effects; sunscreens; or mixtures thereof, and titanium dioxide.

28. The kit of claim 27, wherein the second vehicle comprises di(ethylene glycol) ethyl ether, isopropyl myristate or a mixture thereof.
